(19) United States
(12) Patent Application Publication
(10) Pub. No.: US 2006/0024259 A1
(43) Pub. Date: Feb. 2, 2006

(54) Cosmetic composition comprising, in a non-fatty medium, at least one linear sulfonic polyester and at least one nonionic thickening polymer, processes using this composition and uses thereof

(76) Inventors: Sabine Vrignaud, Clichy (FR); Ludivine Laurent, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/177,333

(22) Filed: Jul. 11, 2005

Related U.S. Application Data
(60) Provisional application No. 60/620,336, filed on Oct. 21, 2004.

(30) Foreign Application Priority Data
Jul. 29, 2004 (FR) 04 08398

(51) Int. Cl. A61K 8/85

(52) U.S. Cl. 424/70.11

(57) ABSTRACT

Disclosed herein is a cosmetic composition comprising, in a non-fatty cosmetically acceptable medium, at least one water-dispersible linear sulfonic polyester and at least one nonionic thickening polymer. Also disclosed herein are a method for shaping, holding, and/or fixing a hairstyle and a method for creating a water-resistant hairstyle by applying the disclosed composition to the hair.

This application claims benefit of U.S. Provisional Application No. 60/620,336, filed Oct. 21, 2004, the contents of which are incorporated herein by reference. This application also claims benefit of priority under 35 U.S.C. § 119 to French Patent Application No. 04 08398, filed Jul. 29, 2004, the contents of which are also incorporated by reference.

Disclosed herein is a cosmetic composition comprising, in a non-fatty cosmetically acceptable medium, at least one water-dispersible linear sulfonic polyester and at least one nonionic thickening polymer. Also disclosed herein are processes using this composition. Further disclosed herein is a method for fixing a hairstyle comprising applying to hair a cosmetic composition comprising, in a non-fatty cosmetically acceptable medium, at least one water-dispersible linear sulfonic polyester and at least one nonionic thickening polymer.

Cosmetic compositions for shaping and/or holding the hairstyle may be in the form of spray compositions comprising a solution, which is generally alcoholic, and at least one component, known as a fixing component, which is generally chosen from polymer resins, the function of which is to form welds between the hairs or to coat the hairs. The fixing component is often formulated as a mixture with various cosmetic adjuvants.

These cosmetic compositions for shaping and/or holding the hairstyle are generally packaged either in a pump-dispenser bottle or in a suitable aerosol container pressurized using a propellant, the aerosol system comprising a liquid phase (or fluid) and a propellant. Styling compositions in a form chosen from gels, creams, and mousses may also be used.

Once such compositions are applied to the hair, it is intended that the fixing compounds therein will make it possible to fix the hair. However, the polymers commonly used as fixing agents in styling compositions do not allow the shape of the hairstyle to be maintained when the hair comes into contact with water for a prolonged period, for instance, when brought into contact with rain or perspiration or during bathing, for example, bathing in the sea or in a swimming pool, and the like.

The present inventors have discovered that the use of a cosmetic composition comprising, in a cosmetically acceptable medium, at least one water-dispersible linear sulfonic polyester and at least one nonionic thickening polymer, makes it possible to fix and/or shape the hairstyle and/or maintain the shape of the hairstyle when the hair comes into contact with water for a prolonged period. This phenomenon is referred to herein as “water resistance”.

As used herein, the term “prolonged period” means contact with water for a minimum period of one minute, for example, 10 minutes or 20 minutes.

The compositions of the present disclosure may also make it possible to obtain styling that is resistant to atmospheric moisture.

The compositions of the present disclosure may allow good fixing and/or good hold of the hair, i.e., a styling effect that lasts throughout the day, or even for several days, and/or shows good water resistance, such as good resistance to repeated bathing. These compositions may also have the advantage of being removed by shampooing. In addition, these compositions may also allow good cosmetic properties to be imparted to the hair.

Disclosed herein therefore is a cosmetic composition comprising, in a non-fatty cosmetically acceptable medium, at least one water-dispersible linear sulfonic polyester and at least one nonionic thickening polymer.

Also disclosed is a method for shaping and/or holding the hairstyle in which the cosmetic composition according to the present disclosure is applied to the hair.

Further disclosed herein is the use of a cosmetic composition comprising, in a non-fatty cosmetically acceptable medium, at least one water-dispersible linear sulfonic polyester and at least one nonionic thickening polymer, as a styling composition for fixing and/or holding the hair, for example, when it comes into contact with water for a prolonged period, i.e., in the case of rain, perspiration, or during bathing, such as bathing in the sea or in a swimming pool. According to this embodiment of the present disclosure, the use of the composition allows a water-resistant hairstyle (shaped hair) to be obtained, such that the fixing or hold of the hair is maintained after the hair comes into contact with water for a prolonged period.

The compositions disclosed herein may be in any form chosen from lotions, sprays, mousses, gels, creams, and pastes.

Other subjects, characteristics, aspects and advantages of the presently disclosed compositions and methods will emerge more clearly upon reading the description and the examples that follow.

As used herein, the term “styling cosmetic composition” means a composition for shaping or holding the hairstyle.

Cosmetically-Acceptable Medium

The cosmetically acceptable medium used in the compositions of the present disclosure is a non-fatty aqueous, alcoholic, or aqueous-alcoholic medium optionally containing at least one additional organic solvent.

Examples of alcohols which may be used in the compositions according to the present disclosure include, but are not limited to, monohydroxylated alkanols chosen from C₁-C₄ lower alcohols, for instance, ethanol, isopropanol, tert-butanol, and n-butanol. In at least one embodiment, the alcohol is ethanol.

The alcohol content in the compositions according to the present disclosure may range from 0 to 50%, for example, from 0 to 10%, or from 0 to 5% by weight relative to the total weight of the composition.

Non-limiting examples of additional organic solvents that may be used in the compositions according to the present disclosure include polyols, for instance, propylene glycol, polyol ethers, and mixtures thereof.

The amount of additional organic solvent in the compositions according to the present disclosure may range from 0 to 30%, for example, from 0 to 20% by weight relative to the total weight of the composition.

As used herein, the term “non-fatty medium” means that the weight amount of fatty substance(s) present in the medium ranges from 0 to 10%, for example, from 0 to 5% by weight relative to the total weight of the composition.

Suitable fatty substances that may be used in the compositions according to the present disclosure may be chosen from silicone and non-silicone fatty substances, for example, silicone and non-silicone mineral and plant oils; waxes; and mixtures thereof.

As used herein, the term “fatty substance” is also considered as being any liposoluble compound, for example, any liposoluble polymer, included in the fatty phase.

Non-limiting examples of silicone oils that may be used in the compositions of the present disclosure include volatile and non-volatile polymethylsiloxanes containing a linear or cyclic silicone chain, which are liquid or pasty at room temperature, for example, cyclopolydimethylsiloxanes (cyclomethicones) such as cyclohexasiloxane; polydimethylsiloxanes comprising at least one group chosen from alkyl, alkoxy, and phenyl groups, which are pendent or at the end of a silicone chain and contain from 2 to 24 carbon atoms; phenylsilicones, for instance, phenyltrimethicones, phenyldimethicones, phenyltrimethylsiloxydiphenylsiloxanes, diphenyldimethicones, diphenylmethyldiphenyltrisiloxanes, 2-phenylethyltrimethyl siloxysilicates, and polymethylphenylsiloxanes; and mixtures thereof.

Suitable non-silicone fatty substances that may be used in the compositions of the present disclosure include all natural or synthetic, organic or mineral, non-silicone oils, waxes, and resins.

As used herein, an “oil” is a lipophilic compound that is liquid at room temperature (about 25° C.), with a reversible solid/liquid change of state. Animal oils and plant oils comprise propane-1,2,3-triol triesters as essential constituents.

Examples of oils that may be used in the compositions of the present disclosure include, but are not limited to, the following:
hydrocarbon-based oils of animal origin, such as perhydrosqualene;
hydrocarbon-based oils of plant origin, such as liquid triglycerides of fatty acids containing from 4 to 10 carbon atoms, for instance, heptanoic and octanoic acid triglycerides; sunflower oil; maize oil; soybean oil; marrow oil; grapeseed oil; sesame oil; hazelnut oil; apricot oil; macadamia oil; arara oil; sunflower oil; castor oil; avocado oil; caprylic/capric acid triglycerides, for instance, those sold by the company Stearineries Dubois or those sold under the names MIGLYOL 810, 812, and 818 by the company Dynamit Nobel; jojoba oil; and shea butter oil;
synthetic esters and synthetic ethers, for example, synthetic esters and ethers of fatty acids, such as oils of formulae R⁶COOR⁷ and R⁶OR⁷ in which R⁶ is a fatty acid residue containing from 8 to 29 carbon atoms and R⁷ is a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyidodecyl erucate, and isostearyl isostearate; hydroxylated esters such as isostearyl lactate, octyl hydroxystearate, octyidodecyl hydroxystearate, diisostearyl malate, triisocetyl citrate, fatty alkyl heptanoates, fatty alkyl octanoates, and fatty alkyl decanoates; polyol esters, for instance, propylene glycol dioctanoate, neopentyl glycol diheptanoate, and diethylene glycol diisononanoate; and pentaerythritol esters, for instance, pentaerythrityl tetraisostearate;
linear or branched hydrocarbons of mineral or synthetic origin, such as volatile or non-volatile liquid paraffins and derivatives thereof; petroleum jelly; polydecenes; and hydrogenated polyisobutene such as parleam oil;
fluid fatty alcohols containing from 8 to 26 carbon atoms, for instance octyldodecanol, 2-butyloctanol, oleyl alcohol, linoleyl alcohol, and linolenyl alcohol;
alkoxylated fatty alcohols, for example, ethoxylated fatty alcohols such as oleth-12;
partially hydrocarbon-based fluoro oils, for instance those described in document JP-A-2 295 912; perfluoromethylcyclopentane and perfluoro-1,3-dimethylcyclohexane, sold under the names “Flutec PC1®” and “Flutec PC3®” by the company BNFL Fluorochemicals; perfluoro-1,2-dimethylcyclobutane; perfluoroalkanes such as dodecafluoropentane and tetradecafluorohexane, sold under the names “PF 5050” and “PF 5060®” by the company 3M, and bromoperfluorooctyl sold under the name “Foralkyl®” by the company Atochem; nonafluoromethoxybutane sold under the name “MSX 4518®” by the company 3M and nonafluoroethoxyisobutane; and perfluoromorpholine derivatives, such as the 4-trifluoromethylperfluoromorpholine sold under the name “PF 5052®” by the company 3M.

As used herein, the term “hydrocarbon-based oil” means any oil predominantly comprising carbon and hydrogen atoms, and possibly ester, ether, fluoro, carboxylic acid, and/or alcohol groups.

As used herein, a “wax” is a lipophilic compound that is solid at room temperature (about 25° C.), with a reversible solid/liquid change of state, having a melting point of greater than about 40° C. and which may be up to 200° C., and having an anisotropic crystal organization in the solid state. As essential constituents, the animal and plant waxes comprise carboxylic acid esters of long-chain alcohols. In general, the size of the wax crystals is such that crystals scatter and/or diffuse light, giving the composition comprising them a more or less opaque, cloudy appearance. By raising the wax to its melting point, it is possible to make it miscible with oils and to form a microscopically homogeneous mixture, but on returning the temperature of the mixture to room temperature, a microscopically and macroscopically detectable recrystallization of the wax in the oils of the mixture is obtained (opalescence).

Non-limiting examples of waxes that may be used in the composition of the present disclosure include waxes of animal origin such as beeswax, spermaceti, lanolin wax, and lanolin derivatives; plant waxes such as sunflower wax, rice wax, potato wax, carnauba wax, candelilla wax, ouricury wax, Japan wax, cocoa butter, cork fiber wax, and sugarcane wax; mineral waxes, for example, paraffin wax, petroleum jelly wax, lignite wax, microcrystalline waxes, ceresin, and ozokerite; synthetic waxes such as polyethylene waxes and Fischer-Tropsch waxes; and mixtures thereof.

Water-Dispersible Linear Sulfonic Polyesters

The compositions according to the present disclosure comprise at least one water-dispersible linear sulfonic polyester. As used herein, the term “water-dispersible linear sulfonic polyester” means any sulfonic polyester capable of forming a dispersion, i.e., a two-phase system in which the first phase is formed from finely divided particles uniformly distributed in the second phase, which is the continuous phase.

As used herein, the term “sulfonic polyester” means copolyesters obtained by polycondensation of at least one dicarboxylic acid or an ester thereof, of at least one diol, and of at least one sulfoaryidicarboxylic difunctional compound substituted on the aromatic nucleus with a group —SO₃M in which M is chosen from hydrogen and metal ions such as Na⁺, Li⁺, and K⁺.

The water-dispersible linear sulfonic polyesters used herein have a weight-average molecular mass ranging from 1,000 to 60,000, for example, from 4,000 to 20,000, as determined by gel permeation chromatography (or GPC).

The glass transition temperature (T_{g}) of these sulfonic polyesters ranges from 10° C. to 100° C. In at least one embodiment of the present disclosure, the T_{g} of the at least one water-dispersible linear sulphonic polyester is greater than or equal to 50° C. The glass transition temperature (T_{g}) is measured by differential thermal analysis (DSC, differential scanning calorimetry) according to ASTM standard D3418-97.

Examples of suitable sulfonic polyesters are described in more detail, for example, in U.S. Pat. Nos. 3,734,874, 3,779,993, 4,119,680, 4,300,580, 4,973,656, 5,660,816, 5,662,893, and 5,674,479.

Sulfonic polyesters which may be used in the compositions of the present disclosure may comprise at least some units derived from isophthalic acid, sulfoaryldicarboxylic acid salts, and diethylene glycol. In at least one embodiment of the present disclosure, the sulfonic polyesters used may be obtained from isophthalic acid, the sodium salt of sulfoisophthalic acid, diethylene glycol, and 1,4-cyclohexanedimethanol.

Non-limiting examples of sulfonic polyesters include those known under the INCI name diglycol/CHDM/isophthalates/SIP, and sold under the trade names “Eastman AQ Polymer” (AQ35S, AQ38S, AQ55S and AQ48 ULTRA) by the company Eastman Chemical.

The at least one water-dispersible linear sulfonic polyester may be present in the compositions according to the present disclosure in an amount ranging from 0.1% to 40%, for example, from 1% to 30%, or from 5% to 25% by weight relative to the total weight of the composition.

Nonionic Thickening Polymers

The compositions according to the present disclosure comprise at least one nonionic thickening polymer.

Suitable nonionic thickening polymers, also known as “rheology modifiers”, may be chosen from fatty acid amides, for example, coconut monoethanolamide, coconut diethanolamide, and oxyethylenated carboxylic acid alkyl ether monoethanolamide; cellulose-based thickeners, such as hydroxymethylcellulose, hydroxyethylcellulose, hydroxypropylcellulose, and hydroxypropylmethylcellulose; guar gum and its derivatives, for instance, the hydroxypropyl guar gum sold under the name JAGUAR HP105 by the company Rhodia; gums of microbial origin, for example, xanthan gum and scleroglucan gum); and nonionic associative polymers.

Non-limiting examples of nonionic associative polymers include:
(1) celluloses modified with groups comprising at least one fatty chain, for example:
   hydroxyethylcelluloses modified with groups comprising at least one fatty chain, such as alkyl, arylalkyl, and alkylaryl groups, and mixtures thereof, wherein the alkyl groups may be C₈-C₂₂ alkyl groups, for instance, the product Natrosol Plus Grade 330 CS® (C₁₋₆ alkyls) sold by the company Aqualon, or the product Bermocoll EHM 100® sold by the company Berol Nobel, and
   celluloses modified with alkylphenyl polyalkylene glycol ether groups, such as the product Amercell Polymer HM-1500® (nonylphenyl polyethylene glycol (15) ether) sold by the company Amerchol;
(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhône-Poulenc;
(3) copolymers of vinylpyrrolidone and of fatty-chain hydrophobic monomers; for example:
   the products Antaron V216® or Ganex V216® (vinylpyrrolidone/hexadecene copolymer) sold by the company I.S.P; and
   the products Antaron V220® or Ganex V220® (vinylpyrrolidone/eicosene copolymer) sold by the company I.S.P;
(4) copolymers of C₁-C₆ alkyl methacrylates or acrylates and of amphiphilic monomers comprising at least one fatty chain, for example, the oxyethylenated methyl acrylate/stearyl acrylate copolymer sold by the company Goldschmidt under the name Antil 208®;
(5) copolymers of hydrophilic methacrylates or acrylates and of hydrophobic monomers comprising at least one fatty chain, for example, polyethylene glycol methacrylate/lauryl methacrylate copolymer.
(6) polyurethane polyethers comprising in their chain both hydrophilic blocks, for example, polyoxyethylenated blocks, and hydrophobic blocks, which may be aliphatic sequences alone and/or cycloaliphatic and/or aromatic sequences;
(7) polymers with an aminoplast ether skeleton containing at least one fatty chain, such as the Pure Thix® compounds sold by the company Sud-Chemie.

In at least one embodiment of the present disclosure, the polyurethane polyethers may comprise at least two hydrocarbon-based lipophilic chains containing from 6 to 30 carbon atoms, separated by a hydrophilic block, the hydrocarbon-based chains possibly being pendent chains, or chains at the end of the hydrophilic block. At least one of the hydrocarbon-based chains may be a pendent chain. In addition, the polymer may comprise a hydrocarbon-based chain at one end or at both ends of a hydrophilic block.

The polyurethane polyethers may be in multiblock form, for instance, in triblock form. Hydrophobic blocks may be at each end of the chain, for example, a triblock copolymer with a hydrophilic central block, or distributed both at the ends and in the chain, for example, a multiblock copolymer. These polymers may also be graft polymers or starburst polymers.

The nonionic fatty-chain polyurethane polyethers may be triblock copolymers in which the hydrophilic block is a polyoxyethylenated chain comprising from 50 to 1,000 oxyethylene groups. Nonionic polyurethane polyethers comprise a urethane linkage between the hydrophilic blocks, from which they derive their name.

Also included among the nonionic fatty-chain polyurethane polyethers are those in which the hydrophilic blocks are linked to the lipophilic blocks via other chemical bonds.

Non-limiting examples of nonionic fatty-chain polyurethane polyethers that may be used in the compositions of the present disclosure include Rheolate 205® containing a urea function, sold by the company Rheox; Rheolate® 208, 204, and 212; Acrysol RM 184®; Elfacos T210® containing a C₁₂₁₄ alkyl chain sold by Akzo; Elfacos T212® containing a C₁₈ alkyl chain, sold by Akzo; and DW 1206B® from Rohm & Haas, containing a C₂₀ alkyl chain and a urethane linkage, sold at a solids content of 20% in water.

It is also possible to use solutions or dispersions of these polymers, for example, in water or in aqueous-alcoholic mediums. Examples of such polymers include Rheolate® 255, Rheolate® 278, and Rheolate® 244 sold by the company Rheox and DW 1206F and DW 1206J sold by the company Rohm & Haas.

Further examples of polyurethane polyethers that may be used in the compositions of the present disclosure include those described in the article by G. Fonnum, J. Bakke and Fk. Hansen—Colloid Polym. Sci 271, 380-389 (1993).

In at least one embodiment of the present disclosure, the compositions may comprise a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Examples of such polyurethane polyethers include those sold by Rohm & Haas under the names Aculyn 44® and Aculyn 46®. Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%). Aculyn 44® is a polycondensate of polyethylene glycol containing 150 or 180 mol of ethylene oxide, of decyl alcohol and of methylenebis(4-cyclohexyl-isocyanate) (SMDI), at 35% by weight in a mixture of propylene glycol (39%) and water (26%).

The at least one nonionic thickening polymer may be present in the compositions according to the present disclosure in an amount ranging from 0.01% to 10%, for example, from 0.05% to 5%, or from 0.1% to 2% by weight relative to the total weight of the composition.

In one embodiment of the present disclosure, the water-dispersible linear sulfonic polyester/nonionic thickening polymer ratio may range from 2% to 50%, for example, from 2.5% to 20%, or from 3% to 15%.

The compositions according to the present disclosure may also contain at least one additional cosmetic adjuvant such as those mentioned below.

Fixing Polymers

For example, as an additional fixing polymer, any anionic, cationic, amphoteric, or nonionic fixing polymer, and mixtures thereof used in the art may be used in the compositions according to the present disclosure.

The at least one additional fixing polymer may be soluble or insoluble in the cosmetically acceptable medium, and may be used in the form of dispersions of solid or liquid polymer particles (latices or pseudolatices).

Anionic Fixing Polymers

The anionic fixing polymers useful herein may be polymers comprising groups derived from acids chosen from carboxylic acid, sulfonic acid, and phosphoric acid and may have a number-average molecular mass ranging from 500 to 5,000,000.

The carboxylic groups may be provided by unsaturated monocarboxylic or dicarboxylic acid monomers such as those corresponding to formula (I): 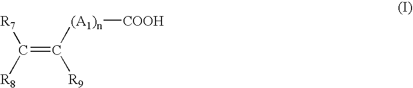
in which
n is an integer from 0 to 10,
A₁ is a methylene group, optionally connected to the carbon atom of the unsaturated group, or to the neighbouring methylene group when n is greater than 1, via a hetero atom such as oxygen or sulfur,
R₇ is chosen from hydrogen, phenyl groups, and benzyl groups,
R₈ is chosen from hydrogen, lower alkyl groups, and lower carboxyl groups, and
R₉ is chosen from hydrogen, lower alkyl groups, —CH₂—COOH, phenyl groups, and benzyl groups.

In the abovementioned formula, a lower alkyl group denotes, for example, a group having 1 to 4 carbon atoms such as methyl and ethyl groups.

Suitable anionic fixing polymers may be chosen from homopolymers and copolymers of acrylic and methacrylic acid and salts thereof, crotonic acid copolymers, copolymers of C₄-C₈ monounsaturated carboxylic acids and anhydrides, polyacrylamides containing carboxylate groups, homopolymers and copolymers containing sulfonic groups, anionic polyurethanes, and anionic grafted silicone polymers.

Examples of anionic fixing polymers containing carboxylic groups include, but are not limited to:
A) Acrylic or methacrylic acid homopolymers and copolymers, and salts thereof, for example, the products sold under the names Versicol® E and K by Allied Colloid and Ultrahold® sold by BASF, copolymers of acrylic acid and of acrylamide sold in the form of their sodium salts under the names RETEN 421, 423, and 425 by Hercules, and sodium salts of polyhydroxycarboxylic acids.
B) Copolymers of acrylic or methacrylic acid with a monoethylenic monomer, such as ethylene, styrene, vinyl esters, acrylic and methacrylic acid esters, optionally grafted onto a polyalkylene glycol, such as polyethylene glycol, and optionally crosslinked. Such polymers are described, for example, in French Patent No. 1 222 944 and German Patent Application No. 2 330 956, the copolymers of this type comprising an optionally N-alkylated and/or hydroxyalkylated acrylamide unit in their chain as described, for instance, in Luxembourg patent application Nos. 75370 and 75371, or sold under the name Quadramer by the company American Cyanamid. Further examples include copolymers of acrylic acid and of C₁-C₄ alkyl methacrylate and terpolymers of vinylpyrrolidone, of acrylic acid, and of methacrylate of C₁-C₂₀ alkyl, for example of lauryl, such as the product sold by ISP under the name Acrylidone® LM, and methacrylic acid/ethyl acrylate/tert-butyl acrylate terpolymers such as the product sold under the name Luvimer® 100 P by BASF. Still further examples include methacrylic acid/acrylic acid/ethyl acrylate/methyl methacrylate copolymers as an aqueous dispersion, sold under the name Amerhold® DR 24 by the company Amerchol.
C) Crotonic acid copolymers, such as those comprising vinyl acetate or propionate units in their chain and optionally other monomers such as allylic esters or methallylic esters, vinyl ethers or vinyl esters of a linear or branched saturated carboxylic acid with a long hydrocarbon chain such as those containing at least 5 carbon atoms, it being possible for these polymers optionally to be grafted or crosslinked, or alternatively another vinyl, allylic or methallylic ester monomer of α- or β-cyclic carboxylic acids. Such polymers are described, inter alia, in French Patent Nos. 1 222 944, 1 580 545, 2 265 782, 2 265 781, 1 564 110, and 2 439 798. Commercial products falling into this class include the resins 28-29-30, 26-13-14, and 28-13-10 sold by National Starch.
D) Copolymers derived from C₄-C₈ monounsaturated carboxylic acids or anhydrides chosen from:
   copolymers comprising (i) at least one entity chosen from maleic acids, maleic anhydrdides, fumaric acids, fumaric anhydrides, itaconic acids, and itaconic anhydrides and (ii) at least one monomer chosen from vinyl esters, vinyl ethers, vinyl halides, phenylvinyl derivatives, and acrylic acid and its esters, the anhydride functions of these copolymers optionally being monoesterified or monoamidated. Such polymers are described in U.S. Pat. Nos. 2,047,398, 2,723,248, and 2,102,113 and British Patent No. 839 805. Commercial products include those sold under the names Gantrez® AN or ES by the company ISP; and
   copolymers comprising (i) at least one entity chosen from maleic, citraconic, and itaconic anhydride units and (ii) at least one monomer chosen from allylic and methallylic esters, optionally comprising at least one group chosen from acrylamide, methacrylamide, α-olefin, acrylic ester, methacrylic ester, acrylic acid, methacrylic acid, and vinylpyrrolidone groups in their chain, the anhydride functions of these copolymers optionally being monoesterified or monoamidated.

These polymers are described, for example, in French Patent Nos. 2 350 384 and 2 357 241 to L'Oreal.
E) Polyacrylamides comprising carboxylate groups.

Examples of homopolymers and copolymers comprising sulfonic groups include, but are not limited to, polymers comprising vinylsulfonic, styrenesulfonic, naphthalenesulfonic, and acrylamidoalkylsulfonic units.

These polymers may be chosen, for example, from:
polyvinylsulfonic acid salts having a molecular mass ranging from 1,000 to 100,000; copolymers with an unsaturated comonomer, such as acrylic or methacrylic acids and their esters; acrylamide or its derivatives; vinyl ethers; and vinylpyrrolidone;
polystyrenesulfonic acid salts such as the sodium salts that are sold, for example, under the names Flexan® 500 and Flexan® 130 by National Starch. These compounds are described, for example, in French Patent No. 2 198 719;
polyacrylamidesulfonic acid salts, such as those mentioned in U.S. Pat. No. 4,128,631, for example, polyacrylamidoethylpropanesulfonic acid, sold under the name COSMEDIA POLYMER HSP 1180 by Henkel.

The branched block anionic polymer sold under the name Fixate G-100 by the company Noveon may also be suitable for use as an anionic fixing polymer.

In at least one embodiment of the present disclosure, the anionic fixing polymers may be chosen from acrylic acid copolymers, such as the acrylic acid/ethyl acrylate/N-tert-butylacrylamide terpolymers sold under the name Ultrahold® Strong by the company BASF; copolymers derived from crotonic acid, such as vinyl acetate/vinyl tert-butylbenzoate/crotonic acid terpolymers and the crotonic acid/vinyl acetate/vinyl neododecanoate terpolymers sold under the name RESIN 28-29-30 by the company National Starch; polymers derived from maleic acids, maleic anhydrides, fumaric acids, fumaric anhydrides, itaconic acids, and itaconic anhydrides with vinyl esters, vinyl ethers, vinyl halides, phenylvinyl derivatives, and acrylic acid and esters thereof, such as the methyl vinyl ether/monoesterified maleic anhydride copolymers sold, for example, under the name Gantrez® by the company ISP; the copolymers of methacrylic acid and of methyl methacrylate sold under the name Eudragit® L by the company Rohm Pharma; the copolymers of methacrylic acid and of ethyl acrylate sold under the name Luvimer® MAEX and MAE by the company BASF; the vinyl acetate/crotonic acid copolymers sold under the name LUVISET CA 66 by the company BASF; the vinyl acetate/crotonic acid copolymers grafted with polyethylene glycol sold under the name Aristoflex® A by the company BASF; and the polymer sold under the name FIXATE G-100 by the company Noveon.

For example, the anionic fixing polymers may be chosen from methyl vinyl ether/monoesterified maleic anhydride copolymers sold under the name Gantrez® ES 425 by the company ISP; the acrylic acid/ethyl acrylate/N-tert-butylacrylamide terpolymers sold under the name Ultrahold® Strong by the company BASF; the copolymers of methacrylic acid and of methyl methacrylate sold under the name Eudragit® L by the company Rohm Pharma; the vinyl acetate/vinyl tert-butylbenzoate/crotonic acid terpolymers and the crotonic acid/vinyl acetate/vinyl neododecanoate terpolymers sold under the name RESIN 28-29-30 by the company National Starch; the copolymers of methacrylic acid and of ethyl acrylate sold under the name Luvimer® MAEX and MAE by the company BASF; the vinylpyrrolidone/acrylic acid/lauryl methacrylate terpolymers sold under the name Acrylidone® LM by the company ISP; and the polymer sold under the name FIXATE G-100 by the company Noveon.

Cationic Fixing Polymers

Suitable cationic fixing film-forming polymers that may be used in the composition of the present disclosure may be chosen from polymers comprising primary, secondary, tertiary, and/or quaternary amine groups forming part of the polymer chain or directly attached thereto, and having a molecular weight ranging from 500 to 5,000,000, for example, from 1,000 to 3,000,000.

Non-limiting examples of cationic fixing polymers include homopolymers and copolymers of acrylic and methacrylic esters and amides containing amine functions, cationic polysaccharides; quaternary copolymers of vinylpyrrolidone and of vinylimidazole; and chitosans.

Further examples of cationic fixing polymers include but are not limited to:
(1) homopolymers and copolymers derived from acrylic and methacrylic esters and amides and comprising at least one of the units of the following formulae: 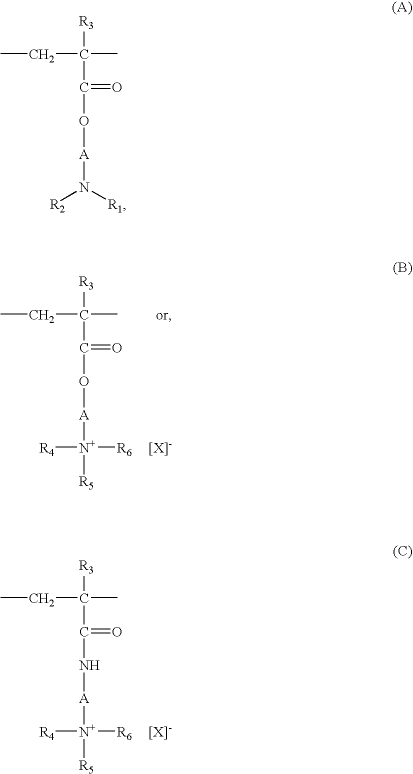
in which:
R₃ is chosen from hydrogen and CH₃;
A is chosen from linear or branched alkyl groups comprising 1 to 6 carbon atoms and hydroxyalkyl groups comprising 1 to 4 carbon atoms;
R₄, R₅, and R₆, which may be identical or different, are chosen from alkyl groups comprising 1 to 18 carbon atoms and benzyl radicals;
R₁ and R₂, which may be identical or different, are chosen from hydrogen and alkyl groups comprising 1 to 6 carbon atoms; and
X is chosen from a methosulfate anion or halides such as chloride and bromide.

The copolymers of family (1) may also contain at least one comonomer unit that may be chosen from acrylamides, methacrylamides, diacetone acrylamides, acrylamides, and methacrylamides substituted on the nitrogen with at least one group chosen from lower (C₁₋₄) alkyl groups, groups derived from acrylic and methacrylic acids and esters thereof, vinyllactams such as vinylpyrrolidone or vinylcaprolactam, and vinyl esters.

Non-limiting examples of copolymers in family (1), include:
copolymers of acrylamide and of dimethylaminoethyl methacrylate quaternized with dimethyl sulfate or with a methyl halide, such as the product sold under the name Hercofloc® by the company Hercules,
copolymers of acrylamide and of methacryloyloxyethyltrimethylammonium chloride, described, for example, in patent application EP-A-0 080 976 and sold under the name BINA QUAT P 100 by the company Ciba Geigy,
copolymers of acrylamide and of methacryloyloxyethyltrimethylammonium methosulfate, such as the product sold under the name Reten by the company Hercules,
quaternized and non-quaternized vinylpyrrolidone/dialkylaminoalkyl acrylate and methacrylate copolymers, such as the products sold under the name “Gafquat®” by the company ISP, such as, for example, “Gafquat® 734” and “Gafquat® 755”, and the products known as “Copolymer® 845, 958, and 937”. These polymers are described, for example, in French Patent Nos. 2 077 143 and 2 393 573,
fatty-chain polymers containing a vinylpyrrolidone unit, such as the products sold under the name STYLEZE W20 and STYLEZE W10 by the company ISP,
dimethylaminoethyl methacrylate/vinylcaprolactam/vinyl pyrrol idone terpolymers, such as the product sold under the name GAFFIX VC 713 by the company ISP, and
quaternized vinylpyrrolidone/dimethylaminopropylmethacrylamide copolymers, such as the products sold under the name “Gafquat® HS 100” by the company ISP;
(2) cationic polysaccharides, for example, cationic polysaccharides containing quaternary ammonium, such as those described in U.S. Pat. Nos. 3,589,578 and 4,031,307, for example guar gums containing trialkylammonium cationic groups. Such products include those that are sold under the trade names JAGUAR C13 S, JAGUAR C 15, and JAGUAR C 17 by the company Meyhall;
(3) quaternary copolymers of vinylpyrrolidone and of vinylimidazole;
(4) chitosans and salts thereof; for example, chitosan acetate, lactate, glutamate, gluconate, and pyrrolidonecarboxylate. For example, mention may be made of chitosan with a degree of deacetylation of 90.5% by weight, such as the product sold under the name KYTAN BRUT STANDARD by the company Aber Technologies, and chitosan pyrrolidonecarboxylate sold under the name Kytamer® PC by the company Amerchol.
(5) cationic cellulose derivatives such as copolymers of cellulose and of cellulose derivatives grafted with a water-soluble monomer comprising a quaternary ammonium, for example, those disclosed in U.S. Pat. No. 4,131,576, such as hydroxyalkylcelluloses, for instance hydroxymethyl-, hydroxyethyl- or hydroxypropylcelluloses grafted with a salt chosen from methacryloyloxyethyltrimethylammonium, methacrylamidopropyltrimethylammonium, and dimethyldiallylammonium salts.

Examples of commercial products corresponding to family (5) include, but are not limited to, the products sold under the name “CELQUAT L 200” and “CELQUAT H 100” by the company National Starch.

Amphoteric Fixing Polymers

Suitable amphoteric fixing polymers that can be used in the composition according to the present invention may be chosen from polymers comprising units B and C distributed randomly in the polymer chain, in which B denotes a unit derived from a monomer comprising at least one basic nitrogen atom and C denotes a unit derived from an acid monomer comprising at least one group chosen from carboxylic and sulfonic groups. Alternatively, B and C may denote groups derived from carboxybetaine or sulfobetaine zwitterionic monomers;

B and C may also denote a cationic polymer chain comprising groups chosen from primary, secondary, tertiary, and quaternary amine groups, in which at least one of the amine groups bears a carboxylic or sulfonic group connected via a hydrocarbon group. Alternatively, B and C may form part of a chain of a polymer containing an α,β-dicarboxylic ethylene unit in which one of the carboxylic groups has been made to react with a polyamine comprising at least one group chosen from primary and secondary amine groups.

Non-limiting examples of suitable amphoteric fixing polymers include copolymers comprising acidic vinyl units and basic vinyl units, crosslinked and acylated polyamino amides, polymers containing zwitterionic units, chitosan-based polymers, modified (C₁-C₅)alkyl vinyl ether/maleic anhydride copolymers, amphoteric polyurethanes, and amphoteric grafted silicone polymers.

Further examples of amphoteric fixing polymers include, but are not limited to, the following polymers:
(1) copolymers having acidic vinyl and basic vinyl units, such as those resulting from the copolymerization of a monomer derived from a vinyl compound bearing a carboxylic group such as acrylic acid, methacrylic acid, maleic acid, and x-chloroacrylic acid, and a basic monomer derived from a substituted vinyl compound containing at least one basic atom, such as, dialkylaminoalkyl methacrylates, dialkylaminoalkyl acrylates, dialkylaminoalkylmethacrylamides, and dialkylaminoalkylacrylamides. Such compounds are described, for example, in U.S. Pat. No. 3,836,537.
(2) polymers comprising units derived from:
   a) at least one monomer chosen from acrylamides and methacrylamides substituted on the nitrogen atom with an alkyl group,
   b) at least one acidic comonomer containing at least one reactive carboxylic group, and
   c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

Examples of suitable N-substituted acrylamides and methacrylamides include compounds in which the alkyl groups comprise from 2 to 12 carbon atoms, for example, N-ethylacrylamide, N-tert-butylacrylamide, N-tert-octylacrylamide, N-octylacrylamide, N-decylacrylamide, N-dodecylacrylamide, and the corresponding methacrylamides.

Suitable acidic comonomers may be chosen from acrylic acid, methacrylic acid, crotonic acid, itaconic acid, maleic acid, fumaric acid, and alkyl monoesters, comprising 1 to 4 carbon atoms, of maleic and fumaric acids and anhydrides.

Examples of basic comonomers include, but are not limited to, aminoethyl, butylaminoethyl, N,N′-dimethylaminoethyl, and N-tert-butylaminoethyl methacrylates.

Copolymers having the CTFA (4th edition, 1991) name octylacrylamide/acrylates/butylaminoethyl methacrylate copolymer, such as the products sold under the name Amphomer® and Lovocryl® 47 by the company National Starch, are also suitable for use in the compositions of the present disclosure.
(3) crosslinked and acylated polyamino amides partially or totally derived from polyamino amides of formula (II): 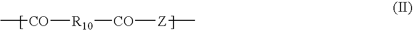
   in which
R₁₀ is chosen from divalent groups derived from saturated dicarboxylic acids, mono- or dicarboxylic aliphatic acids containing an ethylenic double bond, esters of a lower alkanol, comprising 1 to 6 carbon atoms, of these acids and groups derived from the addition of any one of said acids to an amine chosen from bis(primary) and bis(secondary) amines, and
Z is chosen from groups derived from bis(primary), mono- and bis(secondary) polyalkylene-polyamines, for example:
   a) in an amount ranging from 60 to 100 mol %, the group: 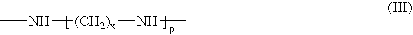
wherein when x=2, and p=2 or 3, and when x=3, p=2
this group being derived from amines chosen from diethylenetriamine, triethylenetetraamine, and dipropylenetriamine;
   b) in an amount ranging from 0 to 40 mol %, the group (III) above, wherein x=2 and p=1 and wherein the group is derived from ethylenediamine, or the group derived from piperazine: 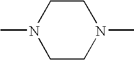
   c) in an amount ranging from 0 to 20 mol %, the —NH(CH₂)₆—NH— group derived from hexamethylenediamine,
these polyamino amides being crosslinked by the addition reaction of a difunctional crosslinking agent chosen from epihalohydrins, diepoxides, dianhydrides, and bis-unsaturated derivatives, using from 0.025 to 0.35 mol of crosslinking agent per amine group of the polyamino amide, and acylated by the action of an entity chosen from acrylic acid, chloroacetic acid, alkane sultones, and salts thereof.

Suitable carboxylic acids may be chosen from acids comprising 6 to 10 carbon atoms, such as adipic acid, 2,2,4-trimethyladipic acid, 2,4,4-trimethyladipic acid, terephthalic acid, and acids containing an ethylenic double bond such as, acrylic acid, methacrylic acid, and itaconic acid.

Examples of alkane sultones which may be used in the acylation may be chosen from sultone and butane sultone. Suitable salts of the acylating agents include the sodium and potassium salts of the acylating agents.
(4) polymers comprising zwitterionic units of the formula: 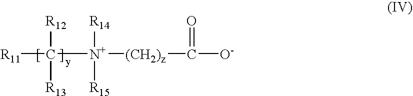
   in which
R₁₁ is a polymerizable unsaturated group such as an acrylate, methacrylate, acrylamide or methacrylamide group,
y and z are independently integers ranging from 1 to 3,
R₁₂ and R₁₃ are independently chosen from hydrogen, methyl, ethyl, and propyl groups,
R₁₄ and R₁₅ are independently chosen from hydrogen and alkyl groups such that the sum of the carbon atoms in R₁₄ and R₁₅ does not exceed 10.

The polymers comprising such units may also comprise units derived from non-zwitterionic monomers, such as dimethyl- and diethylaminoethyl acrylate and methacrylate, alkyl acrylates and methacrylates, acrylamides and methacrylamides, and vinyl acetate.

Examples of such polymers comprising zwitterionic groups include, but are not limited to, the copolymers of methyl methacrylate/methyl dimethylcarboxymethylammonioethyl methacrylate, such as the product sold under the name DIAFORMER Z301 by the company Sandoz.
(5) polymers derived from chitosan comprising monomer units corresponding to the following formulae: 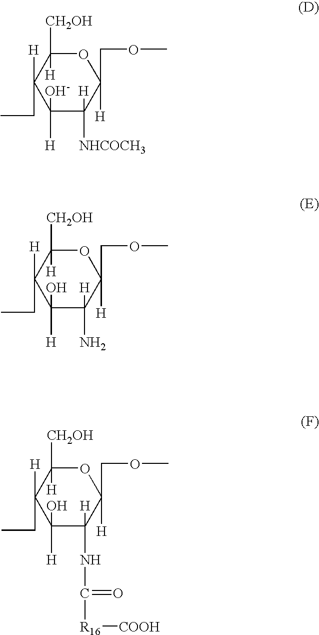
   the unit (D) being present in an amount ranging from 0 to 30%, the unit (E) in an amount ranging from 5% to 50% and the unit (F) in an amount ranging from 30% to 90%, wherein, in unit (F), R₁₆ represents a group of formula: 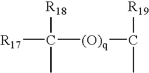
   wherein
when q=0,
   R₁₇, R₁₈, and R₁₉, which may be identical or different, are chosen from hydrogen; methyl, hydroxyl, acetoxy, and amino residues; monoalkylamine residues and dialkylamine residues that are optionally interrupted by at least one nitrogen atom and/or optionally substituted with at least one group chosen from amine, hydroxyl, carboxyl, alkylthio, and sulfonic groups; and alkylthio residues in which the alkyl group bears an amino residue; and
   at least one of the groups R₁₇, R₁₈, and R₁₉ is hydrogen;
when q=1,
   R₁₇, R₁₈, and R₁₉ are hydrogen,
      as well as the salts formed by these compounds with bases or acids.
(6) polymers corresponding to the formula (V), such as those described, for example, in French Patent No. 1 400 366: 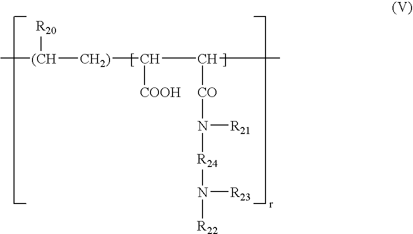
   in which
r is an integer greater than or equal to 1,
R₂₀ is chosen from hydrogen, CH₃O, CH₃CH₂O, and phenyl groups,
R₂₁ is chosen from hydrogen and lower alkyl groups such as methyl and ethyl groups,
R₂₂ is chosen from hydrogen and C₁₋₆ lower alkyl groups such as methyl and ethyl groups,
R₂₃ is chosen from C₁₋₆ lower alkyl groups such as methyl and ethyl groups and groups corresponding to the formula: —R₂₄—N(R₂₂)₂, wherein R₂₄ is chosen from —CH₂—CH₂—, —CH₂—CH₂—CH₂—, and —CH₂—CH(CH₃)— groups and R₂₂ is defined as above, and
R₂₄ is chosen from —CH₂—CH₂—, —CH₂—CH₂—CH₂—, and —CH₂—CH(CH₃)— groups.
(7) polymers derived from the N-carboxyalkylation of chitosan, such as N-carboxymethylchitosan and N-carboxybutylchitosan, sold under the name “EVALSAN” by the company Jan Dekker.
(8) amphoteric polymers of the type -D-X-D-X chosen from:
   a) polymers obtained by the action of chloroacetic acid or sodium chloroacetate on compounds comprising at least one unit of formula (VI):
      -D-X-D-X-D- (VI)
      where D is the group 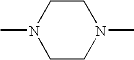
      and X is chosen from the symbols E and E′, which may be identical or different, and represent a divalent group that is an alkylene group with a straight or branched chain containing up to 7 carbon atoms in the main chain, which is unsubstituted or substituted with at least one hydroxyl group and which may comprise, in addition to oxygen, nitrogen, and sulfur atoms, 1 to 3 aromatic and/or heterocyclic rings; the oxygen, nitrogen, and sulfur atoms being present in the form of groups chosen from ether, thioether, sulfoxide, sulfone, sulfonium, alkylamine, alkenylamine, hydroxyl, benzylamine, amine oxide, quaternary ammonium, amide, imide, alcohol, ester, and/or urethane groups;
b) polymers of formula (VI′):
   -D-X-D-X- (VI′)
   where D is the group 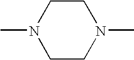
   and X is chosen from the symbols E and E′, wherein
at least one X represents E′;
E has the meaning given above; and
E′ is a divalent group that is an alkylene group with a straight or branched chain having up to 7 carbon atoms in the main chain, which is unsubstituted or substituted with at least one hydroxyl group and containing at least one nitrogen atom, the nitrogen atom being substituted with an alkyl chain that is optionally interrupted by an oxygen atom and necessarily comprising at least one carboxyl function or at least one hydroxyl function and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

In at least one embodiment of the present disclosure, the amphoteric fixing polymer may be chosen from family (3), described above, such as the copolymers having the CTFA name octylacrylamide/acrylates/butylaminoethyl methacrylate copolymer, such as the products sold under the names Amphomer®, Amphomer® LV 71, and Lovocryl® 47 by the company National Starch and family (4), described above, such as the copolymers of methyl methacrylate/methyl dimethylcarboxymethylammonioethyl methacrylate, sold, for example, under the name DIAFORMER Z301 by the company Sandoz.

Nonionic Fixing Polymers

Suitable nonionic fixing polymers that may be used according to the present disclosure may be chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene, and copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, and by the company Hoechst under the name Appretan® N9212;
copolymers of acrylonitrile and of nonionic monomers chosen, for example, from butadiene and alkyl (meth)acrylates; such as the products sold under the name CJ 0601 B by the company Rohm & Haas;
styrene homopolymers;
styrene copolymers, for instance copolymers of styrene and of alkyl (meth)acrylates, such as the products Mowilith® LDM 6911, Mowilith® DM 611, and Mowilith® LDM 6070 sold by the company Hoechst, and the products Rhodopas® SD 215 and Rhodopas® DS 910 sold by the company Rhône-Poulenc; copolymers of styrene, of alkyl methacrylate and of alkyl acrylate; copolymers of styrene and of butadiene; and copolymers of styrene, of butadiene and of vinylpyridine;
polyamides;
vinyl lactam homopolymers other than vinyl pyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF; poly(vinylpyrrolidone/vinyl acetate) copolymers, such as those sold under the name PVPVA® S630L by the company ISP, Luviskol® VA 73, VA 64, VA 55, VA 37, and VA 28 by the company BASF; and poly(vinylpyrrolidone/vinyl acetate/vinyl propionate) terpolymers, for instance, the product sold under the name Luviskol® VAP 343 by the company BASF.

In at least one embodiment of the present disclosure, the alkyl groups of the nonionic fixing polymers may comprise from 1 to 6 carbon atoms.

Grafted Silicone Fixing Polymers

According to the present disclosure, it is also possible to use grafted silicone fixing polymers comprising a polysiloxane portion and a portion consisting of a non-silicone organic chain, one of the two portions constituting the main chain of the polymer, and the other being grafted onto the main chain.

These polymers are described, for example, in patent applications EP-A-0 412 704, EP-A-0 412 707, EP-A-0 640 105, WO 95/00578, EP-A-0 582 152, and WO 93/23009 and U.S. Pat. Nos. 4,693,935, 4,728,571, and 4,972,037.

These polymers may be amphoteric, anionic, or non-ionic. In one embodiment, the polymers are anionic or nonionic.

Such polymers are, for example, copolymers that may be obtained by free radical polymerization from the monomer mixture comprising:
a) 50 to 90% by weight of tert-butyl acrylate;
b) 0 to 40% by weight of acrylic acid;
c) 5 to 40% by weight of a silicone macromer of formula: 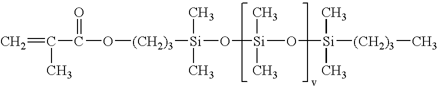
   in which v is a number ranging from 5 to 700, the weight percentages being calculated relative to the total weight of the monomers.

Further examples of grafted silicone polymers include, but are not limited to, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Another non-limiting example of a suitable silicone fixing polymer is the product Luviflex® Silk, sold by the company BASF.

Polyurethane Fixing Polymers

Functionalized or non-functionalized, silicone or non-silicone, cationic, nonionic, anionic or amphoteric polyurethanes, or mixtures thereof may also be used as fixing polymers.

Suitable polyurethanes may also include those disclosed in patent applications EP 0 751 162, EP 0 637 600, EP 0 648 485 and FR 2 743 297, assigned to L'Orèal, patent applications EP 0 656 021 and WO 94/03510, assigned to BASF, and EP 0 619 111, assigned to National Starch.

Other non-limiting examples of polyurethanes that are suitable for the present disclosure include the products sold under the names Luviset Pur® and Luviset® Si Pur by the company BASF.

When the composition according to the present invention comprises at least one additional fixing polymer, the fixing polymer content may range from 0.1% to 20%, for example from 0.5% to 10% by weight relative to the total weight of the composition.

Additional Thickening Polymers

The compositions according to the present disclosure may also contain, as an additional cosmetic adjuvant, at least one additional thickening polymer, also known as a “rheology modifier”. This additional thickening polymer, referred to herein as the co-thickening polymer, may be chosen from cationic, anionic, and amphoteric thickening polymers.

The concentration of the at least one additional thickening polymer, if present, may range from 0.01% to 20%, for example, from 0.05% to 10% by weight relative to the total weight of the composition.

Additives

The styling composition according to the present disclosure may also contain at least one additive chosen from nonionic, anionic, cationic, and amphoteric surfactants; nonionic, anionic, cationic, and amphoteric additional polymers other than the fixing polymers used in the compositions according to the present disclosure; ceramides and pseudoceramides; vitamins and provitamins, including panthenol; silicone and non-silicone water-soluble and liposoluble sunscreens; fillers and solid particles, for instance, coloured and uncoloured mineral and organic pigments; nacreous agents and opacifiers; flakes; active particles; dyes; sequestering agents; plasticizers; solubilizers; acidifying agents; basifying agents; neutralizers; mineral and organic thickeners; antioxidants; hydroxy acids; penetrants; fragrances; and preserving agents.

A person skilled in the art will take care to select the optional additives and the amounts thereof such that they do not harm the properties of the compositions according to the present disclosure.

These additives may be present in the composition according to the instant disclosure in an amount ranging from 0 to 20% by weight relative to the total weight of the composition.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLE 1

Formulation A was prepared by combining the components listed below:

| | % of active |
|---|---|
| Formulation A | material |
| Diglycol/CHDM/isophthalates/SIP copolymer | 20 |
| (Eastman AQ 55 S - Eastman) | |
| Hydroxypropyl guar (JAGUAR HP 105 - Rhodia) | 0.5 |
| Dimethicone (silicone oil) | 1 |
| Stearyl alcohol | 8 |
| Deionized water | qs 100 |
| Fragrance and ingredients | Qs |

Eastman AQ 55 S sold by Eastman Chemical Company is a copolymer of diethylene glycol/1,4-cyclohexanedimethanol/isophthalate/sulfoisophthalate, and is a water-dispersible linear sulfonic polyester as defined herein.

Hydroxypropyl guar (JAGUAR HP 105—Rhodia) is a nonionic thickening polymer as defined herein.

Procedure:

Two grams of the test formulation were applied to a lock of 2.7 g of natural hair 27 cm long. The treated lock was rolled up on a curler 1 cm in diameter, to give it a shape. The product was left to dry in the open air and then the lock was removed delicately from the curler. The lock thus shaped was then immersed in a bath of 8 liters of salt water (3% NaCl) at room temperature, with magnetic stirring at 100 rpm. The length of the lock over time was measured in order to evaluate the shape hold. The results of the shape hold measurements are shown below in Table I. The shape hold was evaluated using the following formula:
% shape hold=(Lᵢ−L)/(Lᵢ−L₀)*100
wherein
L is the length of the curled lock at time t;
L₀ is the length of the curled lock after shaping and removal from the curler; and

Lᵢ is the length of the lock before shaping on the curler.

TABLE I

| | |
|---|---|
| | % shape hold |
| Immersion time | A |

| | | |
|---|---|---|
| 0 | | 100 |
| 35 | seconds | 100 |
| 40 | seconds | 100 |
| 2 | minutes | 100 |
| 5 | minutes | 100 |
| 10 | minutes | 100 |

What is claimed is:

1. A cosmetic composition comprising, in a non-fatty cosmetically acceptable medium, at least one water-dispersible linear sulfonic polyester and at least one nonionic thickening polymer.

2. The cosmetic composition of claim 1, wherein the at least one water-dispersible linear sulfonic polyester is a polycondensate of at least one dicarboxylic acid or ester thereof, of at least one diol and of at least onedifunctional sulfoaryldicarboxylic compound substituted on the aromatic nucleus with a group —SO₃M, wherein M is chosen from hydrogen and metal ions.

3. The cosmetic composition of claim 2, wherein the metal ions are chosen from Na⁺, Li⁺, and K⁺.

4. The cosmetic composition of claim 1, wherein the at least one water-dispersible linear sulfonic polyester is obtained from isophthalic acid, the sodium salt of sulfoisophthalic acid, diethylene glycol, and 1,4-cyclohexanedimethanol.

5. The cosmetic composition of claim 1, wherein the T_{g} of the at least one water-dispersible linear sulfonic polyester is greater than or equal to 50° C.

6. The cosmetic composition of claim 1, wherein the at least one water-dispersible linear sulfonic polyester is present in an amount ranging from 0.1% to 40%, by weight relative to the total weight of the composition.

7. The cosmetic composition of claim 6, wherein the at least one water-dispersible linear sulfonic polyester is present in an amount ranging from 1% to 30% by weight relative to the total weight of the composition.

8. The cosmetic composition of claim 7, wherein the at least one water-dispersible linear sulfonic polyester is present in an amount ranging from 5% to 25% by weight relative to the total weight of the composition.

9. The cosmetic composition of claim 1, wherein the at least one nonionic thickening polymer is chosen from fatty acid amides, cellulose-based thickeners, guar gum and its derivatives, gums of microbial origin, and nonionic associative polymers.

10. The cosmetic composition of claim 9, wherein the fatty acid amide is chosen from coconut monoethanolamide, coconut diethanolamide, and oxyethylenated carboxylic acid alkyl ether monoethanolamide.

11. The cosmetic composition of claim 9, wherein the cellulose-based thickener is chosen from hydroxymethylcellulose, hydroxyethylcellulose, hydroxypropylcellulose, and hydroxypropylmethylcellulose.

12. The cosmetic composition of claim 9, wherein the guar gum derivative is hydroxypropyl guar gum.

13. The cosmetic composition of claim 9, wherein the gum of microbial origin is chosen from xanthan gum and scleroglucan gum.

14. The cosmetic composition of claim 1, wherein the at least one nonionic thickening polymer is chosen from celluloses modified with groups comprising at least one fatty chain; hydroxypropyl guars modified with groups comprising at least one fatty chain; copolymers of vinylpyrrolidone and of fatty-chain hydrophobic monomers; copolymers of C₁₋₆ alkyl acrylates and methacrylates and of amphiphilic monomers comprising at least one fatty chain; copolymers of hydrophilic acrylates and methacrylates and of hydrophobic monomers comprising at least one fatty chain; polyether polyurethanes comprising in their chain both hydrophilic blocks, optionally polyoxyethylenated, and hydrophobic blocks, which may be aliphatic sequences alone and/or cycloaliphatic and/or aromatic sequences; and polymers with an aminoplast ether skeleton containing at least one fatty chain.

15. The cosmetic composition of claim 1, wherein the at least one nonionic thickening polymer is present in an amount ranging from 0.01% to 10% by weight relative to the total weight of the composition.

16. The cosmetic composition of claim 15, wherein the at least one nonionic thickening polymer is present in an amount ranging from 0.05% to 5% by weight relative to the total weight of the composition.

17. The cosmetic composition of claim 16, wherein the at least one nonionic thickening polymer is present in an amount ranging from 0.1% to 2% by weight relative to the total weight of the composition.

18. The cosmetic composition of claim 1, wherein the water-dispersible linear sulfonic polyester/nonionic thickening polymer ratio ranges from 2% to 50%.

19. The cosmetic composition of claim 18, wherein the water-dispersible linear sulfonic polyester/nonionic thickening polymer ratio ranges from 2.5% to 20%.

20. The cosmetic composition of claim 19, wherein the water-dispersible linear sulfonic polyester/nonionic thickening polymer ratio ranges from 3% to 15%.

21. The cosmetic composition of claim 1, further comprising at least one fixing polymer chosen from anionic, cationic, amphoteric, and nonionic fixing polymers, and mixtures thereof.

22. The cosmetic composition of claim 21, wherein the cationic fixing polymer is chosen from homopolymers and copolymers of acrylic and methacrylic esters and amides containing amine functions, cationic polysaccharides, quaternary copolymers of vinylpyrrolidone and of vinylimidazole, and chitosans.

23. The cosmetic composition of claim 21, wherein the anionic fixing polymer is chosen from homopolymers and copolymers of acrylic and methacrylic acids and salts thereof, crotonic acid copolymers, copolymers of C₄-C₈ monounsaturated carboxylic acids and anhydrides, polyacrylamides containing carboxylate groups, homopolymers and copolymers containing sulfonic groups, anionic polyurethanes, and anionic grafted silicone polymers.

24. The cosmetic composition of claim 21, wherein the amphoteric fixing polymer is chosen from copolymers containing acidic vinyl units and basic vinyl units, crosslinked and acylated polyamino amides, polymers containing zwitterionic units, chitosan-based polymers, modified (C₁-C₅)alkyl vinyl ether/maleic anhydride copolymers, amphoteric polyurethanes, and amphoteric grafted silicone polymers.

25. The cosmetic composition of claim 21, wherein the nonionic fixing polymer is chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, nonionic polyurethanes, and nonionic grafted silicone polymers.

26. The cosmetic composition of claim 21, wherein the at least one fixing polymer is present in an amount ranging from 0.1% and 20% by weight relative to the total weight of the composition.

27. The cosmetic composition of claim 26, wherein the at least one fixing polymer is present in an amount ranging from 0.5% to 10% by weight relative to the total weight of the composition.

28. The cosmetic composition of claim 1, further comprising at least one additional thickening polymer chosen from cationic, anionic, and amphoteric thickening polymers, and mixtures thereof.

29. The cosmetic composition of claim 28, wherein the at least one additional thickening polymer is present in an amount ranging from 0.01% to 20% by weight relative to the total weight of the composition.

30. The cosmetic composition of claim 29, wherein the at least one additional thickening polymer is present in an amount ranging from 0.05% to 10% by weight relative to the total weight of the composition.

31. The cosmetic composition of claim 1, further comprising at least one additive chosen from nonionic, anionic, cationic, and amphoteric surfactants; nonionic, anionic, cationic, and amphoteric additional polymers; ceramides and pseudoceramides; vitamins and provitamins; silicone and non-silicone water-soluble and liposoluble sunscreens; fillers and solid particles; nacreous agents and opacifiers; flakes; active particles; dyes; sequestering agents; plasticizers; solubilizers; acidifying agents; basifying agents; neutralizers; mineral and organic thickeners; antioxidants; hydroxy acids; penetrants; fragrances; and preserving agents.

32. The cosmetic composition of claim 31, wherein the fillers and solid particles are chosen from colored and uncolored mineral and organic pigments.

33. A method for shaping, fixing and/or holding a hairstyle comprising applying to hair a composition comprising, in a non-fatty cosmetically acceptable medium, at least one water-dispersible linear sulfonic polyester and at least one nonionic thickening polymer.

34. A method for creating a water-resistant hairstyle, comprising applying to hair a composition comprising, in a non-fatty cosmetically acceptable medium, at least one water-dispersible linear sulfonic polyester and at least one nonionic thickening polymer.
